(19) United States
(12) Patent Application Publication
(10) Pub. No.: US 2013/0190596 A1
(43) Pub. Date: Jul. 25, 2013

(54) Dynamic Optoacoustic Angiography of Peripheral Vasculature

(71) Applicants: Alexander A. Oraevsky, Houston, TX (US); Sergey A. Ermilov, Houston, TX (US)

(72) Inventors: Alexander A. Oraevsky, Houston, TX (US); Sergey A. Ermilov, Houston, TX (US)

(21) Appl. No.: 13/748,563

(22) Filed: Jan. 23, 2013

Related U.S. Application Data
(60) Provisional application No. 61/632,389, filed on Jan. 23, 2012.

(51) Int. Cl. A61B 5/00

(52) U.S. Cl. CPC A61B 5/0095; A61B 5/004
USPC 600/407

(57) ABSTRACT

Provided herein are systems and methods for dynamic optoacoustic imaging of the peripheral vasculature. The system generally comprises a short pulse laser, a fiberoptic laser light delivery system, and imaging module, a scanning system, and electronics/computer system for system control and three-dimensional and two-dimensional optoacoustic image visualization. A method is provided for optoacoustic imaging of an appendage using the dynamic optoacoustic imaging system where an appendage of a subject is imaged under conditions of normal, maximum and minimum tolerable temperatures and displaying differential anatomical images of peripheral vasculature in the appendage and functional diagnostic parameters as a function of time and temperature. From this data medical conditions of the appendage may be diagnosed.

### CROSS-REFERENCE TO RELATED APPLICATIONS

This non-provisional application claims benefit of priority under 35 U.S.C. §119(e) of provisional application U.S. Ser. No. 61/632,389, filed Jan. 23, 2012, now abandoned, the entirety of which is hereby incorporated by reference.

### BACKGROUND OF THE INVENTION

1. Field of the Invention

The present invention generally relates to the relates to the field of biomedical imaging and discloses the designs and methods used in tomography system that can provide medically relevant information about disease of peripheral microvasculature.

2. Description of the Related Art

Peripheral circulation, the distal functional unit of the cardiovascular system, provides exchange sites for gases, nutrients, metabolic wastes, and thermal energy between the blood and the tissues. Pathologic peripheral circulation reflects the breakdown of homeostasis in organisms, which ultimately leads to tissue inevitability (1). In vivo vascular imaging and characterization is of significant physiological, pathophysiological, and clinical importance. The visualization of peripheral vasculature can be useful in diagnostics and monitoring of various medical conditions, like vascular disorders, anatomical abnormalities, and obstructed blood flow (2-4). The examples include stenosis, thrombosis, embolism, frostbite, traumas, peripheral artery occlusive disease, Reynaud's syndrome, etc. The imaging of microcirculation and capillary networks is useful for the diagnosis of ischemia (5).

Another application of the vascular imaging could be related to the physiological stress tests, used to study local vasomotor response. The stress tests involve changing environmental conditions (local occlusion, change of temperature, etc.) in order to bring certain physiological parameters, in this case the peripheral blood flow, close to extreme. Then, the environmental conditions are restored to normal, and the recovery of the individual's peripheral blood flow is monitored (6) Alternatively, the environmental conditions are kept under the stress for a prolonged time, while monitoring the adaptation of the local blood circulation. The patho- and physiologic effects of hypothermal stress are manifested in decreased blood flow caused by vasoconstriction, and reduced tissue metabolism, oxygen utilization, inflammation, and muscle spasm. For every one degree Celsius drop in body temperature, cellular metabolism slows by 5-7%. The patho- and physiologic effects of hyperthermal stress are manifested in increased blood flow caused by vasodilation, activation of tissue metabolism, oxygen utilization, inflammation, and muscle spasm (7).

There are many clinically applicable techniques for localized or systemic heating. Some of the most common involve the use of infrared lasers, focused ultrasound, microwave heating, induction heating, magnetic hyperthermia, infusion of warmed liquids, infrared sauna or direct application of heat such as through sitting in a hot room or wrapping a patient in hot blankets. Acquiring images of tissue throughout stages of response to physiologic stress can allow many conclusions to be made about local and systemic function. However, to ensure that these interpretations have both statistical significance and clinical value, an understanding of how normal physiology would behave within the imaging environment, under the stresses employed during a procedure, must be sought.

Optoacoustic (OA) imaging system is a modality well suitable for visualization of vasculature in live organisms (6-11). It is based on the acoustic signals generated by a short laser pulse absorbed within biological tissues (12). The OA imaging of blood vessels using near infrared (NIR) lasers can be effectively performed at depths up to several centimeters (13). At those laser wavelengths (700-800 nm) blood is 50 to 100 times more absorbing than the surrounding tissues (14), which eliminates the need for use of external optical contrast agents. Currently, microangiography is performed by either MRI or CT (2,4) While these methods have been extensively explored, they have several drawbacks. CT angiography requires contrast agents to distinguish the vasculature. MRI scans are costly, and time required for MRI image acquisition is not optimal for dynamic monitoring of blood flow on short time scales. For comparison, Wang et. al. (3) reports a 3 min 30 s acquisition time.

Thus, there is a need for improved methods for imaging of the peripheral vasculature. Specifically, the prior art is deficient in a system and method for dynamic optoacoustic imaging of peripheral vasculature. The present invention fulfills this longstanding need and desire in the art.

### SUMMARY OF THE INVENTION

The present invention is directed to an optoacoustic imaging system for dynamic angiography. The system comprises means for delivering optical energy to a volume of interest on a subject, means for imaging the volume of interest over a range of temperatures tolerable by the subject and means for producing two- or three-dimensional angiographic images of the volume of interest. The present invention is directed to a related optoacoustic imaging system that further comprises means for ultrasound imaging of the area.

The present invention also is directed to a dynamic optoacoustic angiographic imaging system. The imaging system comprises a short pulse laser operable at one or more wavelengths within a visible and near-infrared spectral range and a fiberoptic laser light delivery system comprising bundles of randomized fibers operatively linked to the laser. The imaging system comprises an imaging module operatively linked to the fiber optic laser light delivery system. The imaging module comprises a vessel having means for supporting an appendage therein, an optoacoustic coupling medium contained within the vessel and covering the appendage, means for precision controlled cooling or heating of the coupling medium, and an array of ultrasonic transducers configured to detect ultrasonic waves generated within the appendage upon delivery of the laser light thereto. The imaging system comprises a rotational and/or translational scanning system operatively linked to the imaging module configured for scanning the ultrasonic transducer array over the appendage. The imaging system comprises an electronics system comprising components for amplification, digitization and processing of the optoacoustic signals acquired from the ultrasonic transducer array and a computer that has a memory, a processor and a display and is in electronic communication with the electronic components and is configured for system control and three-dimensional and two-dimensional optoacoustic image visualization. The present invention also is directed to a related dynamic optoacoustic angiographic imaging system further comprising a system for ultrasound imaging of the appendage.

The present invention is directed further to a method for optoacoustic imaging of an appendage of a subject. The method comprises heating the optoacoustic coupling medium contained within the imaging module comprising the dynamic optoacoustic angiographic imaging system described herein to a normal physiological temperature for the subject and positioning the appendage into the imaging module. An optoacoustic imaging scan of the appendage is performed at the maximum temperature. The temperature of the optoacoustic coupling medium is changed in steps from the normal to a maximum or minimum safe temperature safely tolerable by the subject and an optoacoustic imaging scan is performed at the maximum or minimum safely tolerable temperature. The optoacoustic signals acquired at both the normal and the maximum or minimum safely tolerable temperatures are analyzed and processed and reconstructing two- or three-dimensional optoacoustic images are reconstructed from the analyzed signals.

The present invention is directed to a related method further comprising displaying differential anatomical images of peripheral vasculature in the appendage and functional diagnostic parameters as a function of time and temperature determined from the dynamic optoacoustic angiographic scan of the appendage and diagnosing a medical condition of the appendage or the peripheral vascular system of the subject based on the displayed functional images and diagnostic parameters. The present invention is directed to another related method further comprising performing ultrasonic imaging of the appendage.

Other and further aspects, features, and advantages of the present invention will be apparent from the following description of the presently preferred embodiments of the invention given for the purpose of disclosure.

### BRIEF DESCRIPTIONS OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1B are a schematic of the three-dimensional dynamic optoacoustic microangiography system (LOIS-DMA) in a side cross-sectional view (FIG. 1A) and a bottom view (FIG. 1B).

FIGS. 2A-2C are three-dimensional optoacoustic images of a human finger following the hypothermia stress test at 1 minute (FIG. 2A), 3 minutes (FIG. 2B), 7 minutes (FIG. 2C). The colorbar shows the dynamic range of the implemented linear grayscale palette.

FIGS. 3A-3B depict the radialis indicis (FIG. 3A) and the venal network (FIG. 3B) of the human finger.

FIGS. 4A-4B are three-dimensional optoacoustic frontal images of a live mouse before and during the hypothermal stress test under normal conditions prior to hypothermia showing kidneys, colon, and vascular network in the lower body (FIG. 4A) and after 20 minutes of hypothermia (FIG. 4B).

FIGS. 5A-5B are three-dimensional optoacoustic frontal images of a live mouse before and during the hyperthermal stress test under normal conditions prior to hypothermia showing the vascular network in the lower body (FIG. 5A) and after 15 minutes of hyperthermia (FIG. 5B).

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “optoacoustic imaging” refers to imaging based on optically induced acoustic signals and also is referred to as photoacoustic imaging or thermoacoustic imaging.

As used herein, the term “computer” or “computer system” refers to any networkable tabletop or handheld electronic device comprising a memory, a processor, a display and at least one wired or wireless network connection. As is known in the art, the processor is configured to execute instructions comprising any software programs or applications or processes tangibly stored in computer memory or tangibly stored in any known computer-readable medium.

As used herein, the term “subject” refers to a human or other mammal or animal or to any portion or body part thereof on which imaging, for example, any optoacoustic imaging, as described herein, may be performed.

In one embodiment of the present invention there is provided a optoacoustic imaging system for dynamic angiography comprising means for delivering optical energy to a volume of interest on a subject; means for imaging the volume of interest over a range of temperatures tolerable by the subject; and means for producing two- or three-dimensional angiographic images of the volume of interest. In a further embodiment the optoacoustic imaging system for dynamic angiography comprises means for ultrasound imaging of the volume. In this further embodiment the ultrasound may be electrically generated or may be laser generated. In all embodiments the volume of interest in the subject may be an appendage.

In both embodiments the means for delivering optical energy may comprise a short pulse laser operable at one or more wavelengths and a fiberoptic delivery system optically connected to the laser. Particularly the one or more wavelengths may comprise the visible and near-infrared spectrum where spectral bands are deliverable in a sequence or toggled.

Also in both embodiments the means for imaging the extremity over a range of temperatures may comprise an imaging module having a vessel shaped to receive the volume of interest, an optoacoustic coupling medium contained in the vessel, means for temperature regulation of the coupling medium, and one or more ultrasonic transducers configured to detect ultrasonic waves generated within the volume of interest upon delivery of the optical energy therein and a scanner operatively connected with the imaging module to scan the one or more ultrasonic transducers over the area of interest. Particularly, the scanner may operate in rotational mode or translational mode or a combination thereof and the one or more ultrasonic transducers may comprise a transducer array.

In addition in both embodiments the means for producing two- or three-dimensional angiographic images may comprise electronic components operatively linked to the one or more ultrasonic transducers configured for acquisition of optoacoustic signals, signal and/or image processing and data transmission to a computer and a computer, having a memory, a processor and a display, that is in electronic communication with the electronic components and configured for system control and three-dimensional and two-dimensional optoacoustic image visualization. Particularly, the signal or image processing by electronic components comprises one or more processes that are signal amplification, digitization, filtering, convolution or deconvolution, backprojection, reconstruction, summation, inversion, transformation, or iteration.

In another embodiment of the present invention there is provided a dynamic optoacoustic angiographic imaging system, comprising a) a short pulse laser operable at one or more wavelengths within a visible and near-infrared spectral range; b) a fiberoptic laser light delivery system comprising bundles of randomized fibers operatively linked to the laser; c) an imaging module operatively linked to the fiber optic laser light delivery system, where the imaging module comprises a vessel having means for supporting an appendage therein; an optoacoustic coupling medium contained within the vessel and covering the appendage, means for precision controlled cooling or heating of the coupling medium; and an array of ultrasonic transducers configured to detect ultrasonic waves generated within the appendage upon delivery of the laser light thereto; d) a rotational and/or translational scanning system operatively linked to the imaging module configured for scanning the ultrasonic transducer array over the appendage; e) an electronics system comprising components for amplification, digitization and processing of the optoacoustic signals acquired from the ultrasonic transducer array; and f) a computer, having a memory, a processor and a display, in electronic communication with the electronic components and configured for system control and three-dimensional and two-dimensional optoacoustic image visualization.

In a further embodiment the dynamic optoacoustic angiographic imaging system comprises a system for ultrasound imaging of the appendage. In this ultrasound imaging system the ultrasound may be electrically generated or may be laser generated. In both embodiments the means for supporting the appendage may comprise a bracket.

In yet another embodiment of the present invention there is provided a method for optoacoustic imaging of an appendage of a subject, comprising the steps of a) heating the optoacoustic coupling medium contained within the imaging module comprising the dynamic optoacoustic angiographic imaging system of claim 13 to a normal physiological temperature for the subject; b) positioning the appendage into the imaging module; c) performing an optoacoustic imaging scan of the appendage at the normal temperature; d) changing the temperature of the optoacoustic coupling medium in steps from the normal to a maximum or minimum safe temperature safely tolerable by the subject; e) performing an optoacoustic imaging scan at the maximum or minimum safely tolerable temperature; f) analyzing and processing optoacoustic signals acquired at both the normal and the maximum or minimum safely tolerable temperatures; and g) reconstructing two- or three-dimensional optoacoustic images from the analyzed signals.

Further to this embodiment the method comprises h) displaying differential anatomical images of peripheral vasculature in the appendage and functional diagnostic parameters as a function of time and temperature determined from the dynamic optoacoustic angiographic scan of the appendage; and i) diagnosing a medical condition of the appendage or the peripheral vascular system of the subject based on the displayed functional images and diagnostic parameters. In this further embodiment anatomical and functional details of the peripheral vasculature may characterize thermoregulatory response and other mechanisms responsible for vasomotor activity. In another further embodiment the method comprises performing ultrasonic imaging of the appendage. In all these embodiments the appendage is an arm, a hand, fingers, a leg, a foot, or toes.

Provided herein is a novel laser optoacoustic imaging system (LOIS-DMA) and method for dynamic micro-angiography intended for in vivo vascular imaging of human (animal in general) extremities. The system employs an optoacoustic detector (preferred detector is an array of ultrawide-band ultrasonic transducers) that collects optoacoustic signals around the said extremity (e.g. finger, toe, etc.) providing optoacoustic data necessary for tomographic reconstruction of the three-dimensional images. A near-infrared Q-switched laser is used to generate optoacoustic signals with increased contrast for blood vessels. The laser is coupled through randomized fiberoptic bundles (preferred orientation of illumination is in orthogonal relative to the detector.

This optoacoustic imaging system can be used for diagnostics of various medical conditions that are manifested in change of the peripheral microvasculature in blood flow through extremities. Examples of the medical conditions that could be diagnosed and staged using the LOIS-DMA include the peripheral arterial disease (PAD), diabetic angiopathy, thrombosis, frostbite, and traumas.

Optoacoustic technology has several unique qualities that make it an ideal candidate for integrating physiologic assessment of the circulation with medical imaging. The use of pulsed, near infrared light at powers intended for imaging delivers enough energy to produce an optoacoustic response from light absorption at the molecular level but does not trigger any sensory or systemic responses in the testing subject. In addition, the conditions under which optoacoustic imaging may be performed are very flexible; a testing subject may be imaged in a ventilated room, submerged in water, coated with gels, all at varying temperatures and pressure without inhibiting the imaging mechanics or basic function. The multiplicity of conditions that may be freely employed in optoacoustic imaging can yield great freedom for determining the viability and status of functional tissue.

It is important to observe that changes in peripheral blood perfusion have two primary components: variable arteriole diameter and arteriovenous anastomotic flow. Arteriole diameter is heavily influenced by cardiovascular function and under direct control by the sympathetic nervous system, sensory pathway, and endocrine system. Many variables such as temperature, blood volume, chemical concentrations, and pressure can affect the mechanisms of vasomotor control to promote adaptive vascular changes. The resulting effect either constricts or dilates the arterioles, resulting in both systemic and local changes in the perfusion of tissue. Arteriovenous anastomoses are present throughout the body and have great significance in regards to perfusion during periods of stress, compromised function, or heightened activity.

One type of anastomosis of particular importance is the capillary networks that branch from arterioles in the periphery and viscera. These microscopic networks are the primary sites of nutrient and water exchange between plasma and intracellular fluid, but also have major roles in mediating the effects of vasomotor control. It is important to observe that all capillary arteriovenous anastomoses do not have blood flowing through them at all times. Changes in the many variables affecting vasomotor controls alter the perfusion of these networks. Taken together, arteriovenous anastomoses and arterioles present a very intimate link to the overall homeostasis of an individual.

Using wavelengths of light between 700 nm and 850 nm the optoacoustic imaging is optimized for visualization of the peripheral vasculature. The resulting images produce strong definitions of individual blood vessels, providing the information on varying diameter, branching, and tissue density. The amount of optoacoustic signal generated in tissue depends primarily on the concentration of absorbing species per voxel, here blood, and the amount of light delivered. Assuming a constant amount of light is available, any changes in the magnitude of optoacoustic response from tissue may be attributed to an increase in the concentration blood in tissue, or increased perfusion. The following embodiment of assessing physiology through imaging will outline one of the functions of vasomotor control, biphasic vasodilation in response to hyperthermia.

### Vasomotor Control Assessment: Hyperthermia-induced Activation of Biphasic Vasodilation

A sustained, local hyperthermic stimulus produces a biphasic thermoregulatory response. The biphasic response is composed of fast and delayed mechanisms. The fast response is mediated by the release of CGRP, substance P, and neurokinin from stimulated C-fiber afferent nerves to produce the perception of heat and vasodilation. The fast response initiates a rise in superficial blood flow for approximately 3-5 minutes following sustained heat stimulation. The receptors involved are sensitive to temperatures of 39° C. or greater, but heat perception and the active vasodilation mechanism may have sensitivities down to 29° C., although at the lower end of this range, vasodilation may compete with vasoconstriction tone and be indistinguishable.

The delayed response is mediated by nitric oxide vasodilation. Persistent stimulation beyond the 5 minutes will activate the nitric oxide vasodilatory pathway in order to produce a steady-state exchange of heat across the heated region of skin. Maximal vasodilatory responses occur around 42° C. and require 25-30 minutes to reach steady-state in healthy individuals. Following the fast mechanism, a decline in vasodilation will occur before the delayed response assumes control and climbs to steady-state.

The assessment of vasomotor control should consider the implications of involving the sympathetic nervous system, the sensory pathway, endocrine system, and cardiovascular function. It is also important to observe that the mechanisms of sudomotor control, vasoconstriction, and vasodilation are intimately associated and may produce combined effects following thermo-stimulation. The specific mode of observing vasodilation should produce a specific, localized response to prevent combined thermoregulatory activity. For this assessment, the index finger will be subjected to superficial heat stress while imaged with optoacoustic methods to observe vasodilation. The finger is an ideal candidate as it contains both glabrous and non-glabrous skin on its palmar and dorsal aspects, respectively.

Glabrous skin is known to distinctly lack active vasodilation and may produce vasodilation only via the reduction of sympathetic tone, which produces vasoconstriction, or the release of nitric oxide. Nonglabrous skin does contain active vasodilation and should show the fast mechanism associated with active vasodilation. The following procedure for assessment of hyperthermia-induced activation of biphasic vasodilation is intended to be performed on the index finger within an optoacoustic imaging environment.

Particularly the instant invention demonstrates or provides the following:

### Assessment of Hyperthermia-induced Activation of Biphasic Vasodilation

1. Submerge the region of interest into an imaging environment heated to approximately 25° C. to ensure vasoconstriction is active but without heavy tone. Ensure a comfortable temperature and proper seating for the patient during the scan to prevent activation of extraneous thermoregulatory mechanisms or uncontrolled deviations in sympathetic tone.
2. Image the entire region of interest, taking care to constrain the finger location within the imaging module.
3. Quickly (in 1 minute or less) heat the imaging environment to 40° C. Sustain this temperature until otherwise directed.
4. Once the imaging environment reaches 40° C., immediately begin a second scan to image the entire region of interest. This marks the time of 0, where hyperthermic stimulation begins.
5. Continuously image the entire region of interest for 10 minutes.
6. Wait 5 minutes. At fifteen minutes, image the entire region of interest again.
7. Wait 10 minutes. At 25 minutes, image the entire region of interest again.
8. Finish the hyperthermic stimulation. Quickly reduce the temperature of the imaging environment to 25° C. (in 1 minute or less).
9. Once the imaging environment reaches 25° C., image the entire region of interest again.

### Systemic Sclerosis and Raynaud's Syndrome

Systemic sclerosis, or scleroderma, is a disease resulting from autoimmune destruction of healthy connective tissue throughout the body, including blood vessels and skin. Microangiopathy, affecting the capillary arteriovenous networks, is a primary feature and can be detected early in the course of disease. It is important whether observed symptoms are indeed indicative of systemic sclerosis or due to another disease process.

Raynaud's phenomenon, for example, is a disorder of vasoconstriction frequently involved with systemic sclerosis. Raynaud's can manifest as either a primary condition or secondary to disease such as systemic sclerosis. Distinguishing between the two forms can be difficult as the presentations are similar. In primary Raynaud's, resting hand perfusion is generally very low compared with the early stages of scleroderma, but the distinguishing factor between the two is the response to thermal stress. Specifically, applying hypothermic conditions to a primary Raynaud's subject will result in a rapid loss of perfusion with sustained losses long after restoring temperature to comfortable levels. In systemic sclerosis, the apparent Raynaud's will not have such a profound loss of perfusion and will recover much more quickly after removal of the hypothermic stress.

Considering the protocol for assessment of hyperthermia-induced activation of biphasic vasodilation, it is contemplated that methods may be designed that implement hypothermia to activate vasoconstriction. Arteriole constriction, in contrast to dilation, is constantly active and holds a baseline tone which affects both perfusion of distal branches and blood pressure. Hypothermia generates a heightened level of vasoconstriction to prevent excessive loss of core heat. In primary Raynaud's, the sympathetic tone which defines the level of vasoconstriction is pathologically increased. Systemic sclerosis differs in that the observed loss of perfusion is primarily due to vascular damage.

### Vasomotor Control Assessment: Hypothermia-induced Activation of Vasoconstriction

Other possible thermal stress tests to assess complex aspects of vasomotor control of local temperature include cooling the subject and then heating the subject.

### Image Reconstruction

Continuous 3D imaging with multiple full 360-degree rotations allows implementation of the continuous time-averaged image reconstruction procedure. The procedure involves tomographic reconstruction of the full 360-degree scans sequentially shifted by a single or multiple steps of the rotational motor. The reconstructed 3D images or individual sections of 3D images then form a sequence of frames for the optoacoustic 3D or 2D movie showing the dynamic optoacoustic angiography.

### Dynamic Visualization of Peripheral Vasculature

The present invention demonstrates that it is crucial that the frame rate of the created 3D or 2D optoacoustic movie is standardized by a specific compression ratio with respect to the real frame rate achieved at the moment of angiography procedure. That condition is essential so that all the created optoacoustic movies will be perceived by the doctors in the same manner and could be compared visually across different patients/procedures.

### Spatial Separation of the Microvasculature

The present invention demonstrates a method of spatial separation of the microvasculature and larger peripheral blood vessels for an enhanced diagnostic evaluation. Microvascular networks have different thermal regulation than larger blood vessels. Therefore, they if possible should be separated in optoacoustic images/movies from their larger counterparts. The method of separation may include using optoacoustic transducers with different impulse responses to measure signals from microvasculature and larger vessels. It also may include application of an analog or digital filter to the data already measured by the array of optoacoustic transducers. Lastly, the optoacoustic images/movies can be reconstructed/created and then spatially filtered to get separate information on temporal and spatial behavior of microvasculature and larger vessels during the stress test. The general approach includes retaining lower frequency components for larger blood vessels and higher frequency components for microvasculature.

### Functional Diagnostic Parameters of a Dynamic OA Angiography

Functional diagnostic parameters of dynamic OA angiography are defined separately for microvasculature and larger blood vessels. Visualized extremity/organ is also divided into subsections/compartments, i.e., finger, for example, can be divided into three subsections corresponding to the phalanxes. Then the functional diagnostic parameters are: (a) For each subsection cumulative OA intensity is normalized to its own maximum and is plotted as a function of time. This parameter defines how the visualized subsection or the entire organ is perfused with time. (b) For each subsection cumulative OA intensity is normalized to the OA intensity of the entire organ, and is plotted as a function of time. This parameter tracks spatial distribution of blood as a function of time.

### Applications of LOIS-DMA

It is demonstrated that pathologies associated with vasomotor control are numerous and it is plausible that any pathological state can have some effect. However, the process of establishing the assessment of physiology in imaging should begin with defining how its methods may provide diagnostic or preventative benefit. Building off examples of pathologies that have a strong component of physiologic stress or compromise, which can be assessed using the methods provided here, will both serve to outline the potential of this technology and focus the “window” utilized for diagnosis. Further methods and applications of the optoacoustic imaging under hypothermal/hyperthermal stress are described.

It is demonstrated that the thermoregulatory response and other physiological effects regulated via vasomotor activity could be studied by repetitive three-dimensional optoacoustic imaging following the stress test that modifies the finger blood flow. Future improvements to the stabilization of the finger during the scans will provide less motion artifacts, better resolution and contrast of individual optoacoustic images, which is essential for quantitative differential image analysis and accurate calculation of functional diagnostic parameters. We also plan to increase the speed of each scan and allow uninterrupted multiple scanning, which will allow studying of the faster processes that happen as a result of vasomotor activity.

One skilled in the art of optoacoustic imaging can recognize that such dynamic optoacoustic angiography can be performed in real-time with the invented laser optoacoustic imaging system (LOIS-DMA). Scanning of the probe allows one to locate and track arteries and veins of a human forearm in real-time. This system can be modified to provide fast preoperative mapping of forearm vessels necessary for establishment of hemodialysis. Also, the frame-by-frame analysis of the recorded LOIS-DMA images in a real time movie mode can be used to measure dynamic changes of the cross-section of the arteries. If combined with the Doppler ultrasound, real-time optoacoustic information on the change of the blood vessel crossection provided by LOIS-DMA could be used in estimation of the instantaneous local blood flow rates. Such a localized high resolution analysis of vascular dynamics is superior to traditionally used plethysmography, which provides blood flow information only within a thick segment of the extremity without spatial resolution. Such data would be useful in tests aimed to estimate the physiological response of the peripheral circulatory system to different types of stress, like thermoregulatory mechanisms, vascular effects of the drugs and in diagnosing of local ischemia.

The profile of variations in arterial diameter clearly indicate a cyclical increase and decrease in blood volume that likely reflects the pulsatile nature of arterial blood flow during systole and diastole. This suggests that a real-time optoacoustic system can be potentially used, similarly to plethysmography, for monitoring arterial blood flow during the cardiac cycle and for characterizing properties of vascular wall function, such as elasticity, endothelial function (vascular reactivity), and augmentation index. Clinical conditions that would benefit from such analysis include atherosclerotic peripheral arterial disease, veno-occlusive disease, and venous valvular insufficiency.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

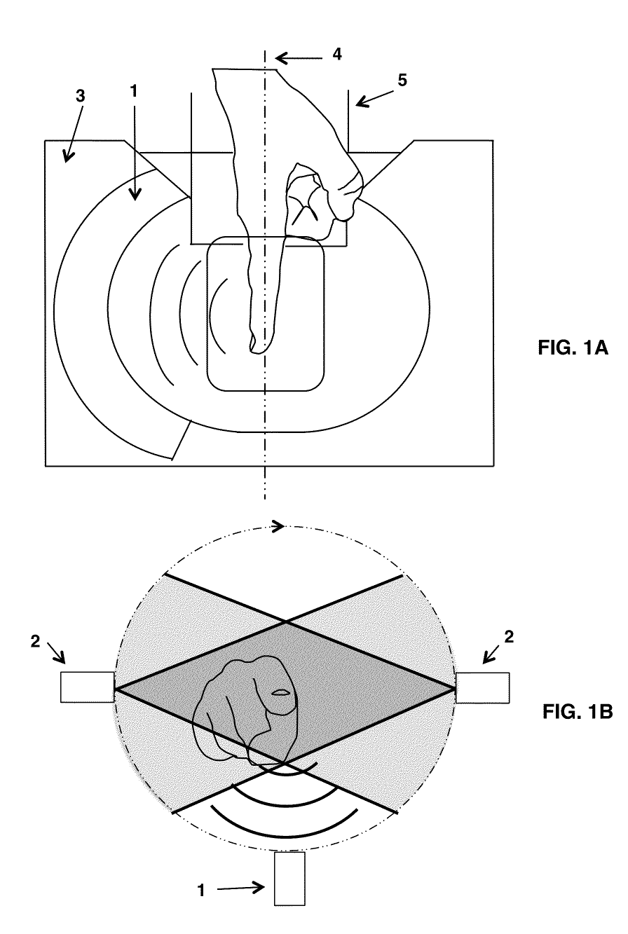

FIG. 1A are an illustration of the three-dimensional optoacoustic microangiography system (LOIS-MA). The LOIS-MA imaging system comprises an acoustic array probe 1, laser fiber bundles 2a,b; a scanning chamber or bowl 3, and a hand bracket 4. A hand 5 of a human subject is positioned within the scanning chamber and held in place by the bracket.

The three-dimensional optoacoustic microangiography system is configured to acquire signals in orthogonal and backward modes of optical illumination. The LOIS-MA system utilizes an array of ultrawide-band ultrasonic transducers which are rotated and/or translated about the object of interest, such as human appendage or extremity. An acoustically dampening chamber containing optoacoustic coupling medium (imaging module) had a volume inside that can be imaged and reconstructed in spherical or cylindrical coordinates. The transducer array and optical fiber bundles were fixed within the imaging module.

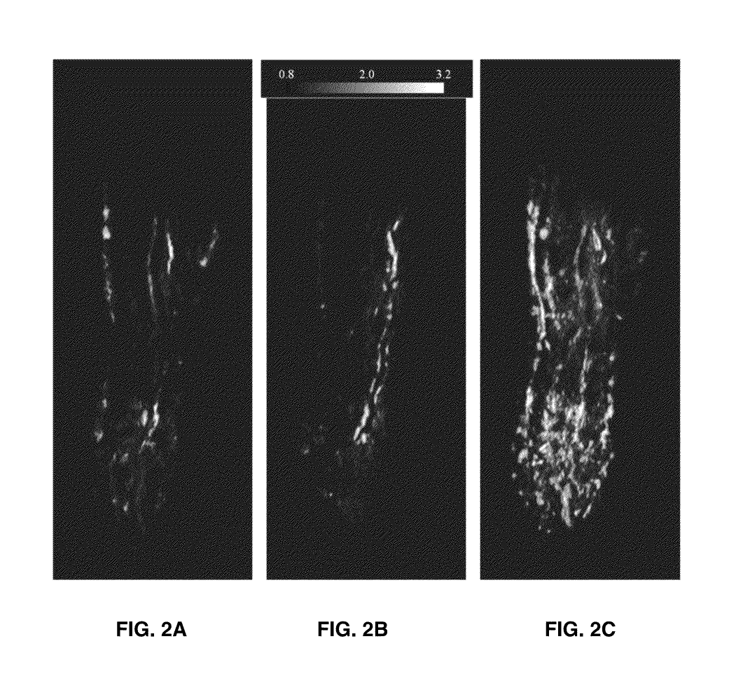

Acoustic signals were detected by an arc array of 128 piezo-composite elements having a frequency bandwidth with maximum frequency corresponding to desirable resolution and minimum detectable frequency corresponding to the largest image feature (tissue structure) to be visualized (as an example, an array with bandwidth of 0.1 MHz to 10 MHz can permit accurate quantitative visualization of tissue structures as large as 10 mm with resolution of about 100 micron. The array aperture was spanning an optimal angular aperture of about 150 deg with a radius sufficient to enclose the object of interest. Illumination was set to be in orthogonal optoacoustic mode coming from a bifurcated, randomized fiber bundle with a rectangular output profile. The imaging module was mounted and centered on the rotational stage operated by a computer controlled motor which would rotate around the object under examination (see FIGS. 2A-2C, 4A-4B and 5A-5B for images of a human finger and a live mouse) for multiple complete 360° scans. The remaining components like the digital acquisition hardware, time gain control, and laser were similar to those of the previously described 3D-OAT mouse imaging system that utilized a water tank as an experimental chamber (6, 13).

FIGS. 2A-2C illustrates dynamic microangiography of a human finger that was performed using the LOIS-MA system with a rotating imaging module. The hypothermia stress test of a human finger included placing the targeted index finger in the ice-cold water for five minutes. Immediately afterwards, the finger was positioned inside the bowl of LOIS-MA and a series of optoacoustic scans were performed approximately 1, 3, 5, and 7 min after the end of the hypothermia stress test. During the last scan, the volunteer reported return of the normal sensory feelings in the finger under study. Each optoacoustic scan continued for about 30 seconds, providing information about thermoregulatory recovery of the peripheral blood flow averaged over that time frame.

Imaging was performed in vegetable oil, which provided acoustic coupling and also reduced the noise picked up by piezoelements of the acoustic array. Temperature of the coupling oil was recorded before and after the imaging procedure and was all the time between 26° C. and 27° C. A full scan consisted of 150 acquisitions with 2.4° steps made without averaging on every other laser pulse. The total scan time was less than 30 seconds. 1536 samples were recorded per acquisition with a sampling frequency of 20 MHz. The amplifier gain was set to 60 dB. During a scan, the index finger of a volunteer was positioned co-linear and in the proximity to the axis of rotation of the bowl. The motion of the finger was restricted at the most proximal, i.e., metacarpophalangeal, joint using a custom-made hand bracket.

Signal conditioning, image processing, and visualization parameters were frozen with respect to the initial scan in order to observe temporal changes. Signal conditioning involved application of a high-pass filter based on a one-scaled wavelet (17) designed to highlight fine optoacoustic features, like small blood vessels and capillaries. Unfortunately, this type of processing also produced speckled imaging noise commonly found in high frequency reconstructions. It was alleviated using a three dimensional Gaussian filter with a standard deviation of 1.5 voxels. 3D optoacoustic images were reconstructed using filtered radial backprojection (6) and visualized using VolView 2.0 (Kitware, Clifton Park, N.Y.). Using the image intensity histogram, all the values of less than pre-defined threshold were made transparent to get rid of the noise. A linear opacity ramp terminating at the opacity level of 0.05 was followed by a stepwise increase in opacity to the level of 0.3 for the remaining data. Color palette was set to a linear 12-bit grayscale. Gradient opacity mapping was handled by the heuristics of the Strong Edge Detection of VolView with the exception of the zero-opacity point, which was set to the mode of the gradient histogram.

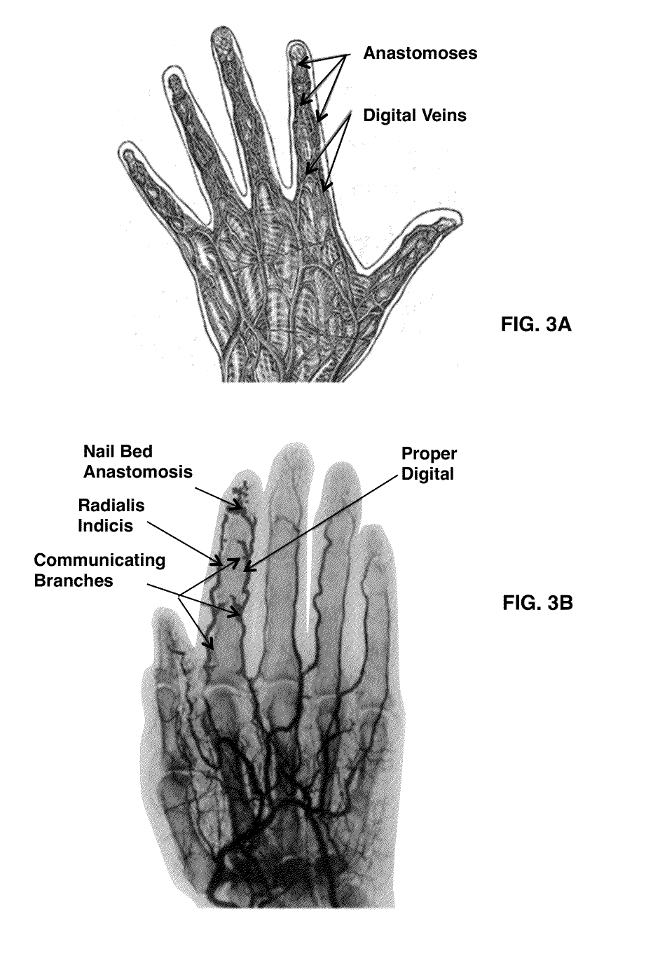

FIGS. 3A-3B depict the vascular anatomy of the human figure. FIG. 3A illustrates the radialis indicis which forms several anastomoses with the proper digital artery. The communicating branches distribute around the joints, supplying structures involved in articulation. The terminal anastomosis found in the nail bed is an important site for regulation of sensory input and thermoregulatory mechanisms. FIG. 3B illustrates the venal network which arises from several anastomoses and converge into proper digital veins that drain via the cephalic vein. Capillary networks, such as the one present in the nail bed are involved in thermoregulation. Vasomotor controls will constrict or increase blood flow through these networks in response to thermal stimuli. The dynamic changes of the local blood flow are dependent on pathological condition of the vasculature.

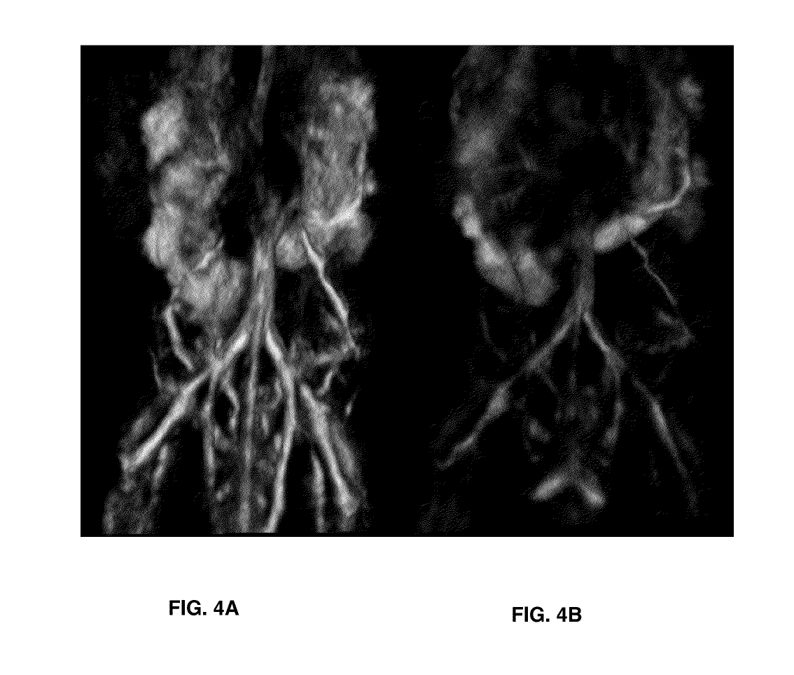

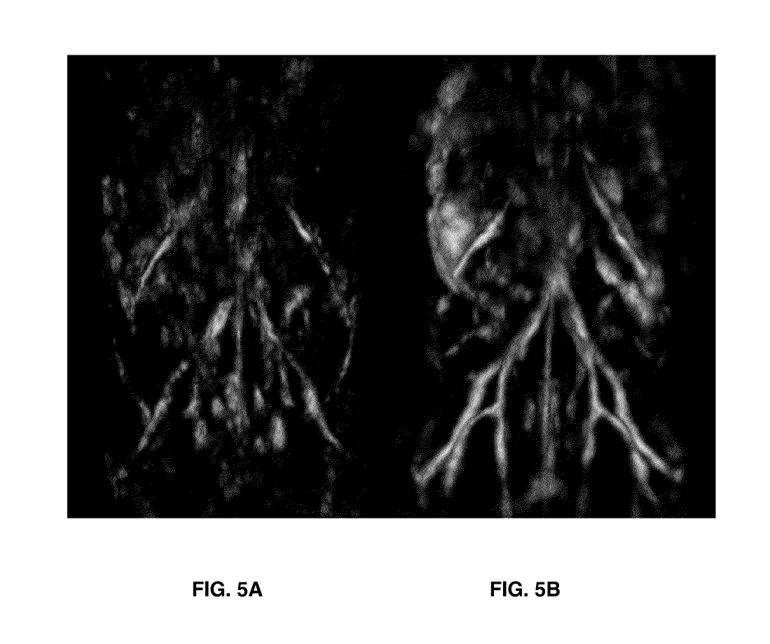

FIGS. 4A-4B and 5A-5B are images Illustrate the results of thermal (hypothermia/hyperthermia) stress tests performed on two mice. Generally, imaging of a mouse was performed in a 7-gallon tank filled with distilled water. Two male Athymic Nude-Foxn1^{nu} mice (Harlan, Indianapolis Ind.) that were 6 to 7 weeks old at the time of the scans were used. They ranged in body mass from 22 to 26 g. Temperature of the water in the tank was continuously monitored with a precision of ±0.1° C. A full scan consisted of ten 360° rotations with 150 acquisitions each, providing a total of 1500 datasets. No averaging was performed during a scan. 1536 samples were recorded per acquisition with a sampling frequency of 20 MHz. The amplifier gain was set to 60 dB. In-vivo mice imaging was done with the use of a mixture of isoflurane and room air that put the mice to sleep and acted as an analgesic. A custom made mouse holder used in our prior studies was used to provide ample imaging area of the body while creating a diving bell for required breathing (20).

In FIGS. 4A-4B the first mouse was subject to a hypothermia stress test. After an initial OA scanning at normal water temperature of 36° C., the temperature control of a tank was turned off and three out of seven gallons of water in the tank were substituted with distilled water at 4° C. After 60 s of manual stirring, the water in the tank was brought to a steady state room temperature of 25° C. The mouse then was scanned continuously for 7 minutes. After 20 minutes of hypothermia, the image shows significant vasoconstriction with reduced optoacoustic intensity and now clearly identified colon.

In FIGS. 5A-5B the second mouse was subject to a hyperthermia stress test. In that after an initial OA scanning at normal water temperature of 36° C., the temperature control of a tank was turned to a maximum level of 45° C. and two out of seven gallons of water in the tank were substituted with boiling water. After 60 s of manual stirring, the water in the tank was brought to a quasi steady state at 45° C. The mouse then was scanned continuously for 7 minutes. Dynamic optoacoustic angiography of a mouse under those thermal stress tests provided information about systemic thermoregulatory response to a moderate short-term hypo- and hyperthermia. After 15 minutes of hyperthermia, the image shows significant vasodilation with increased optoacoustic intensity and now clearly identified portion of the liver.

The following references are cited herein.

1. Hu S. and Wang L.V. J. Biomed. Opt. 2010, 15(1), 011101. doi: 10.1117/1.3281673.
2. Disa et al. Plastic and reconstructive surgery 99(1), 136-144 (1997).
3 Wang, et al. Rheumatology 47(8), 1239-1243 (2008).
4. Toprak et al. Clinical rheumatology 28(6), 679-683 (2009).
5. Met et al. JAMA 301(4), 415-424 (2009).
6. Rosato et al. Rheumatology 50(9), 1654-1658 (2011).
7. Nadler et al. Pain Physician. 2004, 7: 395-399.
8. Brecht et al. Journal of biomedical optics 14(6), 064007 (2009).
9. Fronheiser et al. Journal of biomedical optics 15(2), 021305 (2010).
10. Tsytsarev et al. Journal of biomedical optics 16(7), 076002 (2011).

### 11. Beard, P. Interface Focus 1, 602-631 (2011).

### 12. Oraevsky, A. A. and Karabutov, A. A., “Optoacoustic Tomography”, CRC Press, Boca Raton, 34/31-34/34 (2003).

13. Ermilov et al. Proceedings SPIE 7564, 75641W (2010).
14. Mobley, J. and Vo-Dinh, T. “Optical properties of tissue”, CRC Press, Boca Raton, 2/1-2/75 (2003).
15. Su et al. Proceedings SPIE 7899, 78994B (2011).
16. Wang et al. Proceedings SPIE 6856, 685604 (2008).

### 17. Sun, Y. and Jiang, H. Proceedings SPIE 7258, 72584G (2009).

18. Pozos, R. S. and Danzl, D. F., “Human physiological responses to cold stress and hypothermia”, TMM Publications, Washington, D.C., 351-382 (2002).
19. Ermilov et al. Proceedings SPIE 6856, 685603 (2008).
20. Su et al. Proc. of SPIE 8089, 808902 (2011).

What is claimed is:

1. An optoacoustic imaging system for dynamic angiography comprising:
means for delivering optical energy to a volume of interest on a subject;
means for imaging the volume of interest over a range of temperatures tolerable by the subject; and
means for producing two- or three-dimensional angiographic images of the volume of interest.

2. The optoacoustic imaging system of claim 1, further comprising means for ultrasound imaging of the volume.

3. The optoacoustic imaging system of claim 2, wherein the ultrasound is electrically generated or laser generated.

4. The optoacoustic imaging system of claim 1, wherein the means for delivering optical energy comprises:
a short pulse laser operable at one or more wavelengths; and
a fiberoptic delivery system optically connected to the laser.

5. The optoacoustic imaging system of claim 4, wherein the one or more wavelengths comprise the visible and near-infrared spectrum, and wherein spectral bands are deliverable in a sequence or toggled.

6. The optoacoustic imaging system of claim 4, wherein the means for imaging the extremity over a range of temperatures comprises:
an imaging module having:
a vessel shaped to receive the volume of interest;
an optoacoustic coupling medium contained in the vessel;
means for temperature regulation of the coupling medium; and
one or more ultrasonic transducers configured to detect ultrasonic waves generated within the volume of interest upon delivery of the optical energy therein; and
a scanner operatively connected with the imaging module to scan the one or more ultrasonic transducers over the area of interest.

7. The optoacoustic imaging system of claim 6, wherein the scanner operates in rotational mode or translational mode or a combination thereof.

8. The optoacoustic imaging system of claim 6, wherein the one or more ultrasonic transducers comprise a transducer array.

9. The optoacoustic imaging system of claim 6, wherein the means for producing two- or three-dimensional angiographic images comprises:
electronic components operatively linked to the one or more ultrasonic transducers configured for acquisition of optoacoustic signals, signal and/or image processing and data transmission to a computer; and
a computer, having a memory, a processor and a display, in electronic communication with the electronic components and configured for system control and three-dimensional and two-dimensional optoacoustic image visualization.

10. The optoacoustic imaging system of claim 9, wherein said signal or image processing by electronic components comprises one or more processes that are signal amplification, digitization, filtering, convolution or deconvolution, backprojection, reconstruction, summation, inversion, transformation, or iteration.

11. The optoacoustic imaging system of claim 1, wherein the volume of interest is an appendage of the subject.

12. The optoacoustic imaging system of claim 1, said system configured to evaluate blood flow, ischemia or to assist in administration of hemodialysis.

13. A dynamic optoacoustic angiographic imaging system, comprising:
a) a short pulse laser operable at one or more wavelengths within a visible and near-infrared spectral range;
b) a fiberoptic laser light delivery system comprising bundles of randomized fibers operatively linked to the laser;
c) an imaging module operatively linked to the fiber optic laser light delivery system, said imaging module comprising:
a vessel having means for supporting an appendage therein;
an optoacoustic coupling medium contained within the vessel and covering the appendage,
means for precision controlled cooling or heating of the coupling medium; and
an array of ultrasonic transducers configured to detect ultrasonic waves generated within the appendage upon delivery of the laser light thereto;
d) a rotational and/or translational scanning system operatively linked to the imaging module configured for scanning the ultrasonic transducer array over the appendage;
e) an electronics system comprising components for amplification, digitization and processing of the optoacoustic signals acquired from the ultrasonic transducer array; and
f) a computer, having a memory, a processor and a display, in electronic communication with the electronic components and configured for system control and three-dimensional and two-dimensional optoacoustic image visualization.

14. The optoacoustic imaging system of claim 13, further comprising a system for ultrasound imaging of the appendage.

15. The optoacoustic imaging system of claim 14, wherein the ultrasound is electrically generated or laser generated.

16. The optoacoustic imaging system of claim 13, wherein the means for supporting the appendage comprises a bracket.

17. A method for optoacoustic imaging of an appendage of a subject, comprising the steps of:
a) heating the optoacoustic coupling medium contained within the imaging module comprising the dynamic optoacoustic angiographic imaging system of claim 13 to a normal physiological temperature for the subject;
b) positioning the appendage into the imaging module;
c) performing an optoacoustic imaging scan of the appendage at the normal temperature;
d) changing the temperature of the optoacoustic coupling medium in steps from the normal to a maximum or minimum safe temperature safely tolerable by the subject;
e) performing an optoacoustic imaging scan at the maximum or minimum safely tolerable temperature;
f) analyzing and processing optoacoustic signals acquired at both the normal and the maximum or minimum safely tolerable temperatures; and
g) reconstructing two- or three-dimensional optoacoustic images from the analyzed signals.

18. The method of claim 17, further comprising:
h) displaying differential anatomical images of peripheral vasculature in the appendage and functional diagnostic parameters as a function of time and temperature determined from the dynamic optoacoustic angiographic scan of the appendage; and
i) diagnosing a medical condition of the appendage or the peripheral vascular system of the subject based on the displayed functional images and diagnostic parameters.

19. The method of claim 18, wherein anatomical and functional details of the peripheral vasculature characterize thermoregulatory response and other mechanisms responsible for vasomotor activity.

20. The method of claim 17, further comprising performing ultrasonic imaging of the appendage.

21. The method of claim 17, wherein the appendage is an arm, a hand, fingers, a leg, a foot, or toes.
